(19) United States
(12) Patent Application Publication
(10) Pub. No.: US 2008/0249317 A1
(43) Pub. Date: Oct. 9, 2008

(54) Novel amorphous form of carvedilol phosphate and processes for the preparation thereof

(75) Inventors: Jianguo Wang, Brampton (CA); Bernard Charles Sherman, Toronto (CA)

Correspondence Address: IVOR M. HUGHES, BARRISTER & SOLICITOR,;PATENT & TRADEMARK AGENTS, 175 COMMERCE VALLEY DRIVE WEST, SUITE 200, THORNHILL, ON, L3T 7P6, CA

(73) Assignee: APOTEX INC.

(21) Appl. No.: 11/889,082

(22) Filed: Aug. 9, 2007

Related U.S. Application Data
(60) Provisional application No. 60/910,093, filed on Apr. 4, 2007.

(51) Int. Cl. C07D 209/00

(52) U.S. Cl. 548/414

(57) ABSTRACT

A novel form of amorphous carvedilol phosphate which is particularly suitable for pharmaceutical applications, and processes for preparing said novel form.

### CROSS-REFERENCE TO RELATED APPLICATIONS

This application claims the benefit of U.S. Provisional Application No. 60/910,093, filed Apr. 4, 2007.

### FIELD OF THE INVENTION

The present invention relates to a new amorphous form of carvedilol phosphate and methods for its preparation. This form is particularly well-suited for pharmaceutical applications.

### BACKGROUND OF THE INVENTION

Carvedilol (1,1-(9H-Carbazol-4-yloxy)-3-[[2-(2-methoxyphenoxy)ethyl]amino]-2-propanol) is an antihypertensive which is also effective for the treatment of congestive heart failure and angina. Carvedilol is as nonselective β-adrenoreceptor antagonist and an α₁-adrenoreceptor antagonist having no intrinsic sympathomimetic activity. It is marketed as its racemic free-base under the brand name COREG® by GlaxoSmithKline. In early 2007, a controlled-release form of carvedilol (COREG CR®) is planned to be sold by SB Pharmco which employs the carvedilol phosphate salt form of carvedilol as the active ingredient. “Carvedilol phosphate” will be understood to mean the salt comprising one mole of phosphoric acid per mole of carvedilol (about 0.2411 phosporic acid per gram of carvedilol). This amount of phosphoric acid relative to carvedilol will also be referred to as stochiometric phosphoric acid.

Carvedilol was disclosed originally in U.S. Pat. No. 4,503,067. There are many known polymorphic and pseudopolymorphic forms of carvedilol. For instance, WO 1999/05105, WO 2002/00216, WO 2003/059807 and WO 2006/135757 describe Forms I to VI of crystalline forms of carvedilol. Likewise, US 2006/0148878 teaches various pseudopolymorphic forms of carvedilol.

WO 2004/002419, US 2005/0169994 and US 2006/0182804 teach various salts of carvedilol and/or corresponding solvates. In particular, these include carvedilol phosphate.

The choice of salt form for this product is critical since, for use as a medicine, it is essential to have a form of carvedilol that has sufficient water solubility to ensure good in vivo resorption. For instance, as its hydrochloride salt, which is the protonated form which would be generated in an acidic medium such as gastric fluid, carvedilol exhibits reduced solubility, thereby limiting the bio-adsorption. In this regard, the solubility characteristics of the crystalline carvedilol phosphate taught in WO 2004/002419, US 2005/0169994 and US 2006/0182804 are purportedly superior. However, the possibility of having a more water soluble polymorphic form of carvedilol phosphate while retaining good chemical stability would be especially advantageous.

Given the difficulties associated with finding suitable processes to and forms of carvedilol phosphate, new and industrially acceptable solutions, which offer advantages relative to the prior art, were required.

### SUMMARY OF THE INVENTION

We surprisingly discovered that the addition of a small amount of an acid, for instance acetic acid, greatly assisted the dissolution of carvedilol and stoichiometric phosphoric acid, in a variety of solvents. Even more surprisingly, it allowed the isolation of a previously undisclosed and highly soluble amorphous form of carvedilol phosphate by, for example, dissolution of the carvedilol and stochiometric phosphoric acid in a solvent followed by removal of the solvent by, for instance, spray-drying or concentration.

The improved solubility by the addition of acetic acid is exemplified by the following results. A solution was formed rapidly by adding 1 gram of crystalline carvedilol phosphate, containing 2% acetic acid (w/w relative to the carvedilol), to methanol (3 mL). This may be compared to the same experiment except using crystalline carvedilol phosphate and in the absence of acetic acid wherein about 100 mL of methanol was required for dissolution.

The amorphous form has many desirable characteristics including that it has generally improved solubility in solvents, including methanol, ethyl acetate, and water, relative to the crystalline form. It is also free-flowing, has good chemical stability, retains very little residual solvent, and can be prepared in a highly pure form.

### BRIEF DESCRIPTION OF THE DRAWINGS

The following figures illustrate preferred and alternative embodiments of the invention, wherein:

FIG. 1 shows a PXRD Diffractogram (CuKα radiation) of amorphous carvedilol phosphate produced according to an embodiment of the methods of this invention.

FIG. 2 shows an IR (KBr) Spectrum of amorphous carvedilol phosphate produced according to an embodiment of the methods of this invention.

### DETAILED DESCRIPTION OF THE INVENTION

The amorphous form of carvedilol phosphate may be prepared in various ways. This is due, in part, to the surprisingly improved solubility of carvedilol and stoichiometric phosphoric acid salt, especially in the presence of a small amount of acid. Examples of suitable acids include alkyl, aryl, and aralkyl carboxylic acids and mineral organic acids. Preferred acids are volatile acids including acetic acid and hydrochloric acid, most preferably acetic acid. Preferred amounts of the acid relative to the carvedilol are from 1% to about 20%.

Techniques to prepare amorphous carvedilol phosphate include dissolution of carvedilol and stoichiometric phosphoric acid in a combination a volatile acid, and a second volatile solvent followed by removal of the solvents by concentration using, for example, a rotoevaporater and, optionally, further drying the resulting foam-like solid. The most preferred volatile acid is acetic acid. The most preferred second volatile solvent is a C1 to C4 alkyl alcohol; most preferably the alkyl alcohol is methanol.

In another aspect of the invention, the solvents are removed by spray drying.

For the above processes to prepare amorphous carvedilol phosphate, it is desirable to keep the temperature at about 20° C. to reflux, most preferably about 50° C. to about 55° C., during the preparation.

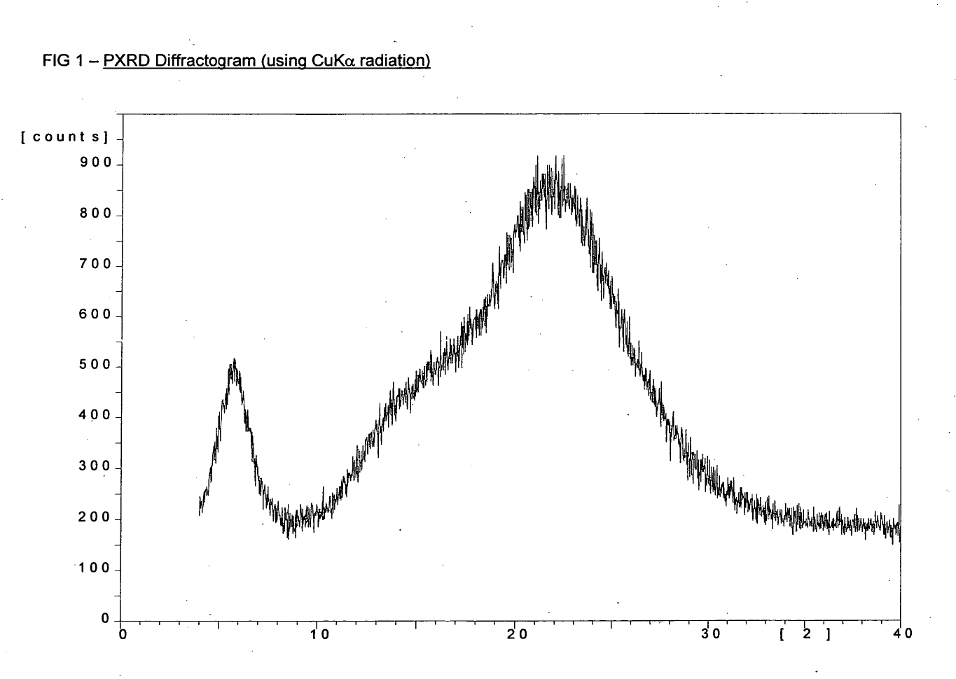

Amorphous carvedilol phosphate prepared by these processes may be characterized by a PXRD pattern as depicted in FIG. 1.

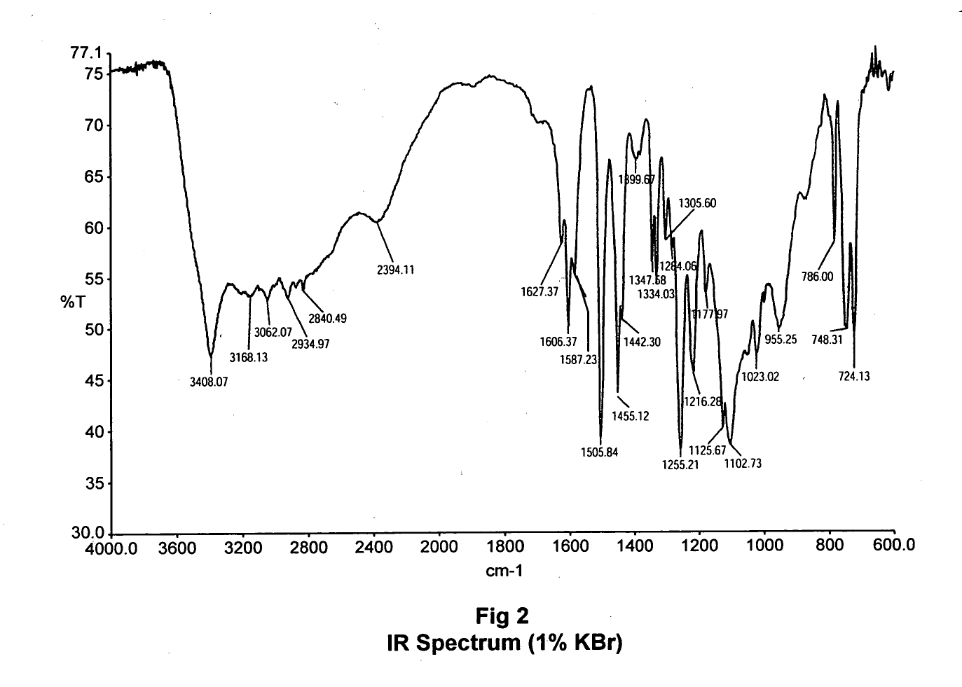

Amorphous carvedilol phosphate prepared by these processes may be characterized by an IR pattern as depicted in FIG. 2.

The amorphous carvedilol phosphate prepared by these processes can be characterized by its IR spectrum (1% KBr) having characteristic peaks expressed in cm⁻¹ at approximately 3408, 1606, 1506, 1455, 1255, 1216 and 724.

The following example is merely representative of the present invention and not intended to be limiting.

### EXAMPLE 1

### Preparation of Amorphous Carvedilol Phosphate

Methanol (12 mL) and 85% phosphoric acid (1.42 g) were added to a round bottom flask. The flask used to add the phosphoric acid was rinsed with another 3 mL portion of methanol which was also added to the round bottom flask. This was followed by the addition of acetic acid (0.1 mL) and the mixture was warmed to 50-55° C. at which point carvedilol free base (5 g) was added with stirring until a clear solution was obtained. The solution was stirred a further 30 minutes at 50-55° C. whereupon the solvents were evaporated using a rotoevaporater (bath temperature=50° C.) to provide a white foam which was dried in a vacuum oven at 55-60° C. for 1 hour. It was then ground to a fine-powder and the powder was dried further in a vacuum oven at 55-60° C. for about 2 hours at which point the residual methanol level was less than 0.3% and the purity was great than 99% by HPLC.

¹H NMR (300 MHz, DMSO-d₆); δ 11.3 (s, 1H); 8.24 (d, J=7.8 Hz, 1H); 7.46 (d, J=8.0 Hz); 7.35-7.30(m, 2H); 7.29-6.85 (m, 6 H); 6.69 (d, J=7.8 Hz); 4.41-4.38(m, 1H); 4.25-4.18 (m, 4H); 3.70(s, 3H); 3.27-3.10 (m, 4H).

The PXRD spectrum of this material is given in FIG. 1 and the IR spectrum is given in FIG. 2.

While the foregoing provides a detailed description of a preferred embodiment of the invention, it is to be understood that this description is illustrative only of the principles of the invention and not limitative. Furthermore, as many changes can be made to the invention without departing from the scope of the invention, it is intended that all material contained herein be interpreted as illustrative of the invention and not in a limiting sense.

1. Amorphous carvedilol phosphate.

2. Highly soluble amorphous carvedilol phosphate.

3. Amorphous carvedilol phosphate characterized by its IR spectrum (1% KBr) having characteristic peaks expressed in cm⁻¹ at approximately 3408, 1606, 1506, 1455, 1255, 1216 and 724.

4. Amorphous carvedilol phosphate having substantially the same IR spectrum as depicted in FIG. 2.

5. Amorphous carvedilol phosphate characterized by substantially the same PXRD diffractogram as depicted in FIG. 1.

6. Amorphous carvedilol phosphate according to claims 3 or 4 further characterized by substantially the same PXRD diffractogram as depicted in FIG. 1.

7. A process of making amorphous carvedilol phosphate comprising the steps of:
i) dissolution of carvedilol and stochiometric phosphoric acid in a combination of a volatile acid and a second volatile solvent, other than water,
ii) removal of the solvents, and
iii) optionally further drying the precipitate.

8. The process of claim 7 wherein the second volatile solvent is a C1 to C4 alcohol.

9. The process of claim 7 wherein the second volatile solvent is methanol.

10. The process of claim 7 wherein the volatile acid is acetic acid.

11. The process of claim 7 wherein the process is conducted at about 20° C. to reflux.

12. The process of any of claims 7 to 11 wherein the reaction is conducted at about 50° C. to about 55° C.

13. The process of claim 10 wherein the amount of acetic acid by weight is from 1% to 20% of the amount of carvedilol.

14. Amorphous carvedilol phosphate made by a process of any of claims 7, 8, 9, 10, 11 or 13.

15. Amorphous carvedilol phosphate made by the process of claim 12.
